(19) United States
(12) Patent Application Publication
(10) Pub. No.: US 2026/0335012 A1
(43) Pub. Date: Oct. 29, 2026

(54) USER INTERFACE AND USER EXPERIENCE FOR AN INTRAVASCULAR LITHOTRIPSY SYSTEM

(71) Applicant: FastWave Medical Inc., Maple Grove, MN (US)

(72) Inventors: Edward ANDERSON, Maple Grove, MN (US); JiChao SUN, Santa Rosa, CA (US); Trevor THIESS, Minneapolis, MN (US); Lauren ENO, Prior Lake, MN (US); Brady HATCHER, Rogers, MN (US); Curtis GOREHAM-VOSS, Maple Grove, MN (US); Tristan TIESO, Minneapolis, MN (US); Andrew LUDWIG, St. Louis Park, MN (US); Scott NELSON, Mission Viejo, CA (US); Emily LAGRANT, Minneapolis, MN (US); Peter YOUNG, Saint Paul, MN (US); Randy BEYREIS, Andover, MN (US); Dean IRWIN, Carlsbad, CA (US)

(21) Appl. No.: 19/733,092

(22) Filed: Jul. 7, 2026

Related U.S. Application Data
(63) Continuation of application No. PCT/US24/57184, filed on Nov. 22, 2024.
(60) Provisional application No. 63/618,763, filed on Jan. 8, 2024.

(51) Int. Cl. A61B 18/22; A61B 17/00; A61B 18/00; A61B 18/20

(52) U.S. Cl. CPC A61B 18/22; A61B 2017/00199; A61B 2018/00172; A61B 2018/00702; A61B 2018/2015

(57) ABSTRACT

Included in the present disclosure is an apparatus, such as an intravascular lithotripsy (IVL) device including a generator. In some embodiments, the generator is configured to provide energy through a catheter. According to some embodiments, the apparatus includes a display coupled to the generator. The apparatus may include a status indicator.

### CROSS-REFERENCE TO RELATED APPLICATIONS

The present application claims priority to PCT Application No. PCT/US2024/057184; filed on Nov. 22, 2024; and entitled IMPROVED USER INTERFACE AND USER EXPERIENCE FOR AN INTRAVASCULAR LITHOTRIPSY SYSTEM.

The present application claims priority to U.S. Provisional Patent Application No. 63/618,763; filed on Jan. 8, 2024; and entitled IMPROVED USER INTERFACE AND USER EXPERIENCE FOR AN INTRAVASCULAR LITHOTRIPSY SYSTEM.

### TECHNICAL FIELD

The present disclosure relates to treatments for a calcified-plaque lesion in a patient’s vasculature.

### BACKGROUND

During an intravascular lithotripsy (IVL) procedure, a clinician uses a catheter configured to break apart calcified-plaque lesions within a patient’s vasculature. Some such methods include the creation and rapid collapse of cavitation bubbles to create a shock wave which causes this calcification break-up.

### SUMMARY

Included in the present disclosure is an apparatus, including a generator configured to provide electricity to a catheter. In some embodiments, the apparatus includes a display coupled to the generator, the display including a status indicator.

According to some embodiments, the catheter is an intravascular lithotripsy (IVL) catheter. The display may be coupled to a remote computing device. In some embodiments, the status indicator is displayed via a software application running on the remote computing device.

According to some embodiments, the status indicator is configured to indicate the apparatus is in a ready state. In some embodiments, the status indicator is configured to indicate the apparatus is in a rest state. The status indicator may be configured to indicate the apparatus is experiencing an error.

According to some embodiments, the display includes a counter. The counter may be configured to count down a number of remaining pulses in a treatment cycle. In some embodiments, the counter is configured to count down a remaining amount of time in a cooldown period.

According to some embodiments, the display includes a pulse counter wheel. The pulse counter wheel may be configured to visually indicate a number of remaining pulses in a treatment cycle. In some embodiments, the pulse counter wheel includes a progress bar. According to some embodiments, the pulse counter wheel is circular.

The display may include a rest counter wheel. In some embodiments, the rest counter wheel is configured to visually indicate a remaining amount of time in a cooldown period. According to some embodiments, the rest counter wheel includes a progress bar. The rest counter wheel may be circular.

In some embodiments, the display includes a remaining pulses indicator. According to some embodiments, the remaining pulses indicator is configured to count down a number of remaining pulses in a life expectancy of the catheter.

The display may include a maximum pulses indicator. In some embodiments, the maximum pulses indicator is configured to indicate a total number of pulses in a life expectancy of the catheter.

According to some embodiments, the display includes a size indicator. The size indicator may include a balloon diameter. In some embodiments, the size indicator includes a balloon length.

Also included in the present disclosure is a system, including an elongated body having an inner elongated structure including a guidewire lumen. In some embodiments, the system includes an erasable programmable read-only memory (EPROM) configured to store information. According to some embodiments, the system includes a balloon positioned at a distal portion of the elongated body, the balloon configured to be inflated. The system may include an optical fiber positioned along the elongated body and within the balloon, the optical fiber configured to emit pressure waves to fragment a buildup in an organ. In some embodiments, the system includes a generator configured to provide energy to the optical fiber, the generator including a display configured to show the information. According to some embodiments, the elongated body is configured to couple to the generator, thereby forming an optical pathway between the generator and the optical fiber.

The generator may be configured to receive power through a direct current (DC) power supply. In some embodiments, the generator includes a female connector, and the elongated body includes a male connector. According to some embodiments, the male connector is configured to couple to the female connector, thereby coupling the elongated body to the generator.

The display may include a status indicator. In some embodiments, the status indicator is configured to indicate the system is in a ready state. According to some embodiments, the status indicator is configured to indicate the system is in a rest state. The status indicator may be configured to indicate the system is experiencing an error.

The display may include a counter. In some embodiments, the counter is configured to count down a number of remaining pulses in a treatment cycle. According to some embodiments, the counter is configured to count down a remaining amount of time in a cooldown period.

The display may include a pulse counter wheel. In some embodiments, the pulse counter wheel is configured to visually indicate a number of remaining pulses in a treatment cycle. According to some embodiments, the pulse counter wheel includes a progress bar. The pulse counter wheel may be circular.

In some embodiments, the display includes a rest counter wheel. According to some embodiments, the rest counter wheel is configured to visually indicate a remaining amount of time in a cooldown period. The rest counter wheel may include a progress bar. In some embodiments, the rest counter wheel is circular.

According to some embodiments, the display includes a remaining pulses indicator. The remaining pulses indicator may be configured to count down a number of remaining pulses in a life expectancy of the elongated body.

In some embodiments, the display includes a maximum pulses indicator. According to some embodiments, the maximum pulses indicator is configured to indicate a total number of pulses in a life expectancy of the elongated body.

The display may include a size indicator. In some embodiments, the size indicator includes a balloon diameter. According to some embodiments, the size indicator includes a balloon length.

The system may include an actuator, the actuator configured to cause the generator to provide electricity to the emitter. In some embodiments, the actuator is configured to send a single pulse to the emitter in response to being activated once. According to some embodiments, the actuator is configured to send a continual stream of pulses to the emitter in response to being activated continuously. The actuator may be configured to send a continual stream of pulses to the emitter in response to being activated twice in quick succession.

In some embodiments, the generator is configured to read the information. According to some embodiments, the generator is configured to write the information.

Also included in the present disclosure is a system, including an elongated body having an inner elongated structure including a guidewire lumen. In some embodiments, the system includes a balloon positioned at a distal portion of the elongated body, the balloon configured to be inflated. According to some embodiments, the system includes an optical fiber positioned along the elongated body and terminating within the balloon, the optical fiber configured to generate pressure waves to fragment a buildup in an organ. The system may include a generator configured to provide energy through the optical fiber, the generator configured to receive power through a direct current (DC) power supply, the generator including a display. In some embodiments, the elongated body is configured to couple to the generator, thereby forming an optical pathway between the generator and the optical fiber.

According to some embodiments, the generator includes a female connector, and the elongated body includes a male connector. The male connector may be configured to couple to the female connector, thereby coupling the elongated body to the generator.

In some embodiments, the system further includes an erasable programmable read-only memory (EPROM) configured to store information. According to some embodiments, the generator is configured to read the information. The generator may be configured to write the information.

In some embodiments, the display includes a status indicator. According to some embodiments, the status indicator is configured to indicate the system is in a ready state. The status indicator may be configured to indicate the system is in a rest state. In some embodiments, the status indicator is configured to indicate the system is experiencing an error.

According to some embodiments, the display includes a counter. The counter may be configured to count down a number of remaining pulses in a treatment cycle. In some embodiments, the counter is configured to count down a remaining amount of time in a cooldown period.

According to some embodiments, the display includes a pulse counter wheel. The pulse counter wheel may be configured to visually indicate a number of remaining pulses in a treatment cycle. In some embodiments, the pulse counter wheel includes a progress bar. According to some embodiments, the pulse counter wheel is circular.

The display may include a rest counter wheel. In some embodiments, the rest counter wheel is configured to visually indicate a remaining amount of time in a cooldown period. According to some embodiments, the rest counter wheel includes a progress bar. The rest counter wheel may be circular.

In some embodiments, the display includes a remaining pulses indicator. According to some embodiments, the remaining pulses indicator is configured to count down a number of remaining pulses in a life expectancy of the elongated body.

The display may include a maximum pulses indicator. In some embodiments, the maximum pulses indicator is configured to indicate a total number of pulses in a life expectancy of the elongated body.

According to some embodiments, the display includes a size indicator. The size indicator may include a balloon diameter. In some embodiments, the size indicator includes a balloon length.

According to some embodiments, the system further includes an actuator, the actuator configured to cause the generator to provide electricity to the emitter. The actuator may be configured to send a single pulse to the emitter in response to being activated once. In some embodiments, the actuator is configured to send a continual stream of pulses to the emitter in response to being activated continuously. According to some embodiments, the actuator is configured to send a continual stream of pulses to the emitter in response to being activated twice in quick succession.

### BRIEF DESCRIPTION OF THE DRAWINGS

These and other features, aspects, and advantages are described below with reference to the drawings, which are intended to illustrate, but not to limit, the invention. In the drawings, like characters denote corresponding features consistently throughout similar embodiments.

FIG. 1 illustrates a diagrammatic view of an intravascular lithotripsy (IVL) system as it may appear inserted into a patient’s vasculature.

FIG. 2 is a diagram illustrating an IVL system.

FIG. 3 illustrates an embodiment of the IVL system of FIG. 2 as it may appear in a use-case scenario.

FIG. 4 illustrates a block diagram of the laser energy source system, such as the generator of FIG. 3, according to some embodiments.

FIG. 5 illustrates a perspective view of an actuator communicatively coupled to a hub, according to some embodiments.

FIG. 6 illustrates a perspective view of a generator, according to some embodiments.

FIG. 7 illustrates an example back view of a generator.

FIG. 8 illustrates an example front view of a generator.

FIG. 9 illustrates the display of FIG. 8, according to some embodiments.

FIG. 10 illustrates an additional embodiment of the display of FIG. 8.

### DETAILED DESCRIPTION

Although specific embodiments are disclosed below, inventive subject matter extends beyond the specifically disclosed embodiments to alternative embodiments and/or uses and modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations, in turn, in a manner that may be helpful in understanding specific embodiments; however, the order of description should not be construed to imply that these operations are order-dependent. Additionally, the structures, systems, and/or devices described herein may be embodied as integrated or separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

During an intravascular lithotripsy (IVL) procedure, a clinician uses the formation and subsequent collapse of cavitation bubbles to generate high-energy pressure waves to disrupt calcified-plaque lesions within a patient’s vasculature. Some such IVL procedures include the generation of shock waves through the providing of laser energy, such as via an optical fiber.

Throughout the present specification, the terms “emit” and “generate” are used synonymously and intended to represent the same action – the causing of or formation of a cavitation bubble and/or pressure wave.

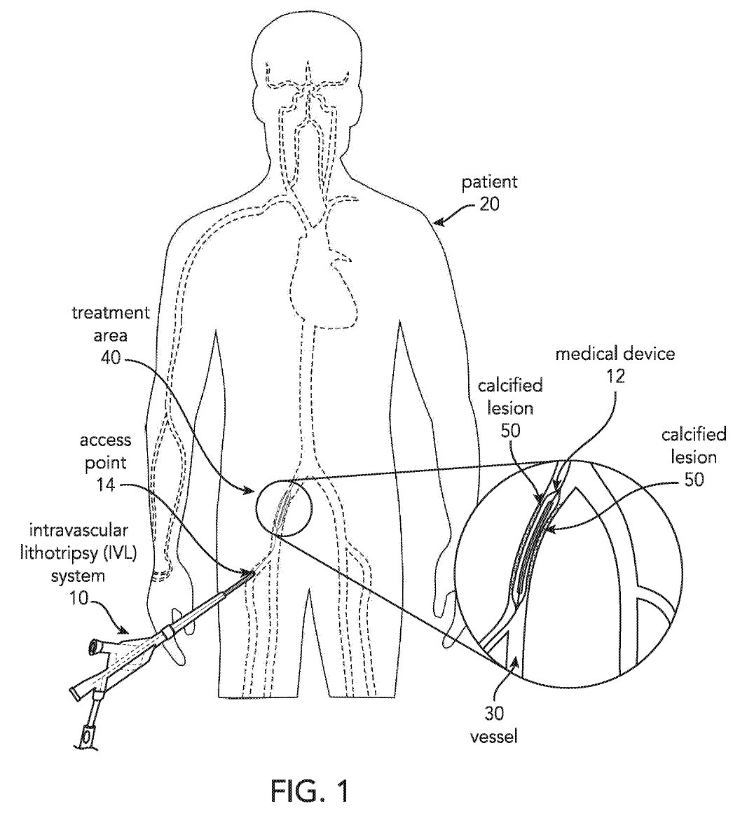

FIG. 1 illustrates a diagrammatic view of an intravascular lithotripsy (IVL) system 10 as it may appear inserted into a patient’s vasculature. The IVL system 10 may include a medical device 12, perhaps including an interventional balloon, as depicted in later figures. During a lesion-disintegration procedure, a clinician may advance the medical device 12 through an access point 14 in the patient 20, such as the femoral or common femoral arteries, as depicted in FIG. 1. Other access points may include the radial artery, tibial artery, pedal artery, axial artery, peroneal artery, etc. The medical device 12 may then be advanced through the vasculature of the patient 20 until it reaches the vessel 30 containing the treatment area 40. For IVL, the treatment area may include a calcified lesion 50. In some embodiments, the treatment area includes coronary arteries.

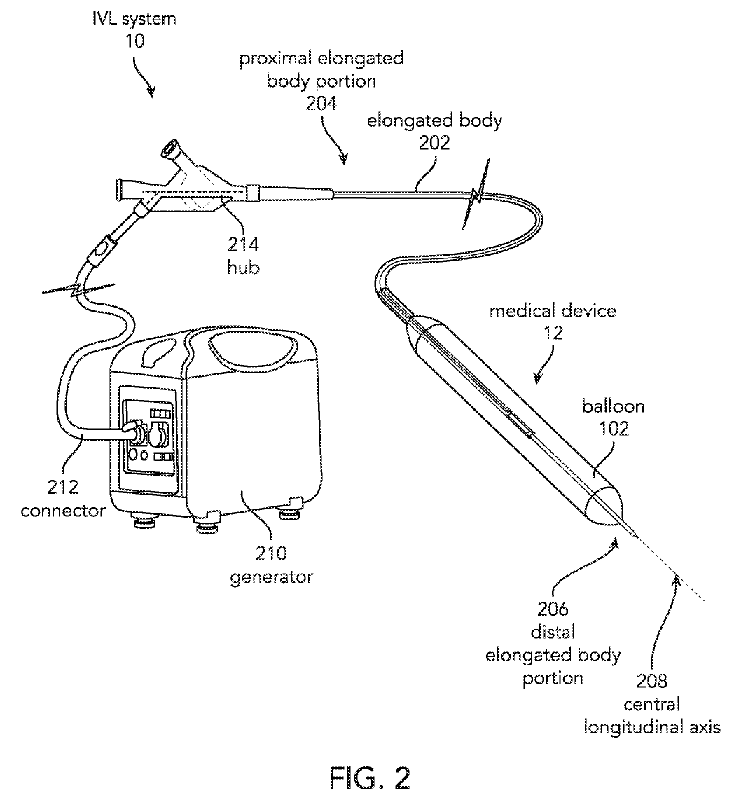

FIG. 2 is a diagram illustrating an IVL system 10. As shown in FIG. 2, IVL system 10 may include at least an energy generator 210 and an elongated body 202 removably coupled to the energy generator 210, such as via an electrical connector 212. The elongated body 202 may include a medical device 12 positioned at a distal elongated body portion 206. In some embodiments, the elongated body 202 is configured to navigate a tortuous vasculature of a patient 20 toward a target treatment site 40, e.g., a calcified-plaque lesion 50 within a vessel 30.

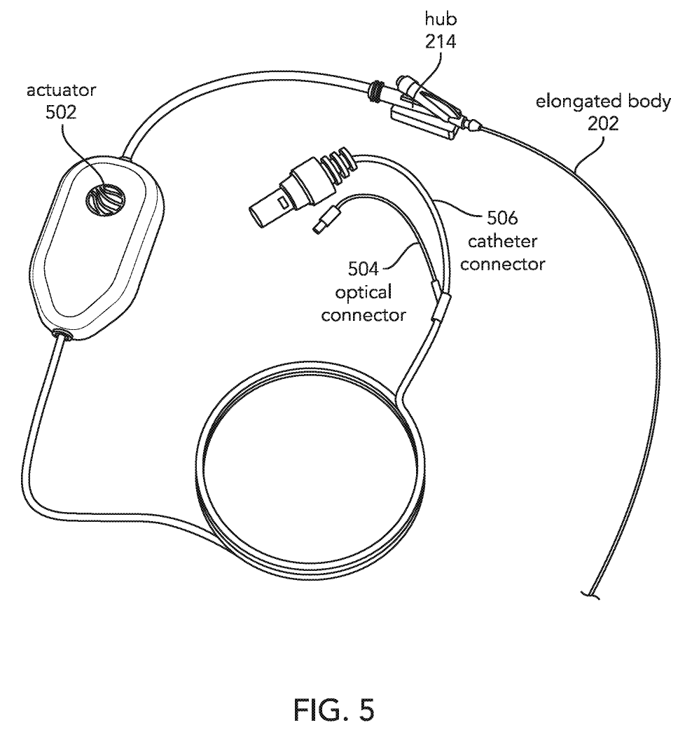

While the term “elongated body” is used throughout the present specification, it is understood that an elongated body 202 may refer to a catheter, such as an IVL catheter. Additionally, while the connector 212 is described as an electrical connector 212 in the description of FIG. 2, it is understood that the connector 212 may also be an optical connector (such as optical connector 504 as seen in FIG. 5). In fact, in some embodiments, the IVL system 10 may not necessitate an electrical connector 212. In these embodiments, a therapy button may be present on the console, and there may be no electrical interrogation of the catheter itself. While an electrical connector 212 may not be necessary in all embodiments, an optical connector is necessary in order to provide the laser energy to the catheter.

As shown in FIG. 2, the medical device 12 may include a fluid-inflatable interventional balloon 102 and a pressure wave emitter array, shown but not labeled due to size constraints, positioned within the balloon 102. The emitter array may include one or more individual emitter units. For instance, the interventional balloon 102, or a distal elongated body portion 206 passing therethrough, may define a central longitudinal axis 208, and emitter units may be distributed longitudinally along the central longitudinal axis 208.

Each emitter unit is configured to receive energy from the energy generator 210 and use the received energy to generate and transmit high-energy pressure waves through the balloon 102 and across a treatment site. As detailed further below, the energy generator 210 may generate and transmit energy in the form of electrical energy, optical energy, or a combination thereof. For instance, the emitter unit(s) may use the received energy to generate a cavitation bubble within the fluid inside the balloon 102, propagating one or more high-energy pressure waves radially outward through the balloon 102 and the calcified lesion 50.

In some cases, but not all cases, a secondary set of high-energy pressure waves can subsequently result from the collapse of the fluid cavitation bubble, further destabilizing the internal structure of the calcified-plaque lesion. In some embodiments, one or more emitters can include an optical-based emitter configured to receive a high-energy optical (e.g., light) signal from the generator 210, such as via one or more optical fibers, and direct the optical signal to trigger the initial cavitation. Additionally, one or more emitters can include an electrical-based emitter configured to receive electrical energy from the generator 210, such as via one or more conductive wires, and generate a spark between a pair of electrodes, thereby triggering the initial cavitation.

According to some embodiments, a cooling mechanism functions in tandem with the energy generator 210. However, flashlamp systems may provide energy to the optical fibers without necessitating said cooling mechanism. Additionally, diode systems may be used as an alternative to flashlamp systems, which may also not require a cooling mechanism.

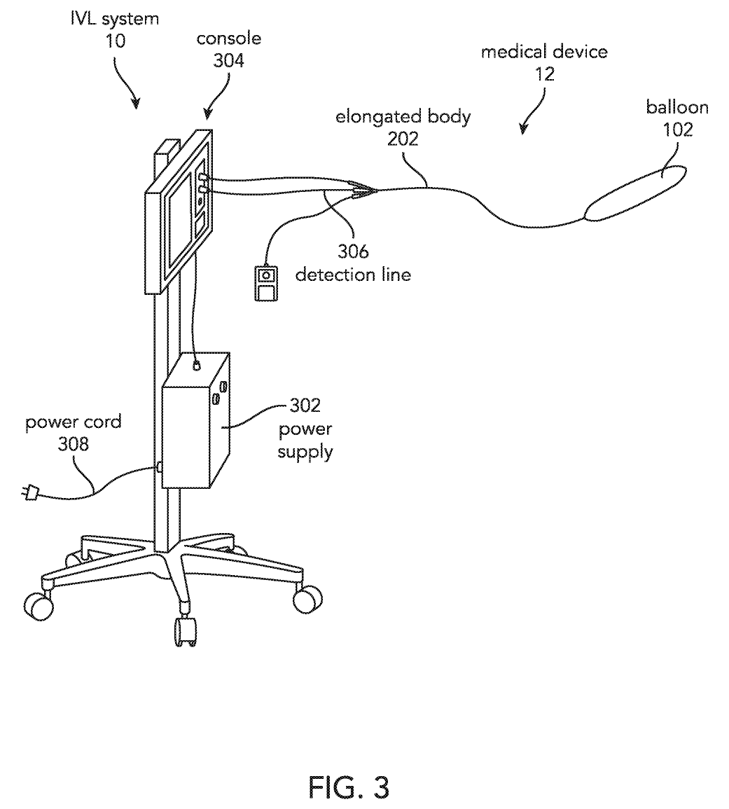

FIG. 3 illustrates an embodiment of the IVL system 10 of FIG. 2 as it may appear in use. A medical post may be provided to facilitate movement of the IVL system 10 between rooms as necessary. A power supply 302 may be situated near the base of the medical post, coupled to a power cord 308 for receiving wall power or power from a generator 210, as well as an umbilicus for electrically coupling to a console 304. As shown in FIG. 3, the console 304 allows a user, such as a clinician, to operate the IVL system 10. The elongated body 202 may be coupled to the console 304 via a power cable for receiving energy from the power supply 302 to transmit energy to the emitters within the IVL balloon 102. A separate line dedicated to the inflation of the IVL balloon 102 may also be present.

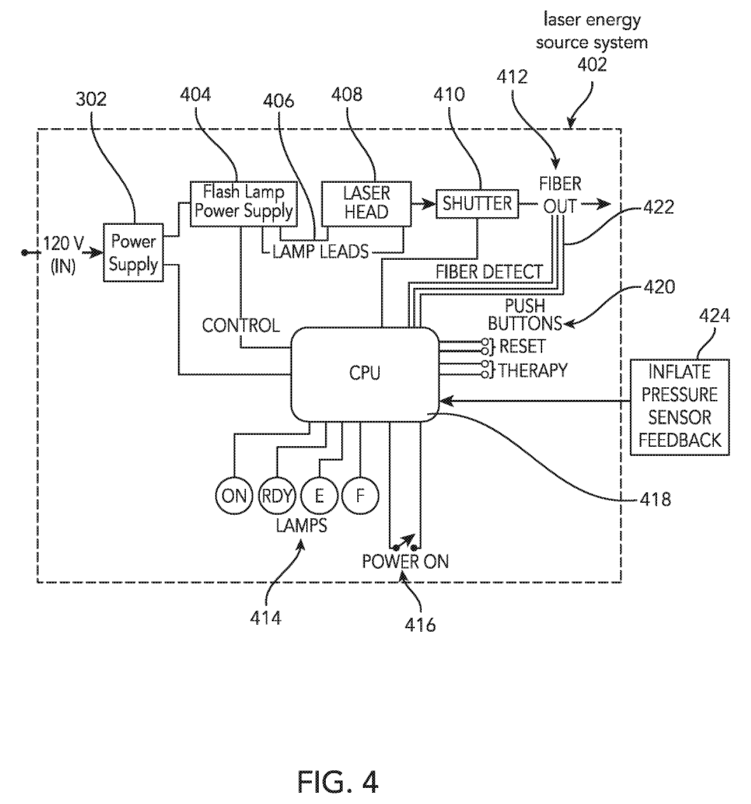

According to some embodiments, a detection line 306 is present. The detection line 306 may offer a few methods of providing feedback about the integrity of the individual components within the IVL balloon 102. For instance, a safety pressure sensor (perhaps the pressure sensor 422 as seen in FIG. 4) may be provided. If a sudden pressure drop is detected, a failure may have occurred, such as a rupture of the IVL balloon 102. This suggests to the clinician that the procedure should be halted, and the IVL balloon 102 should be retrieved immediately and safely from the patient’s vasculature. According to some embodiments, once a pressure sensor in the IVL balloon 102 detects a balloon 102 rupture, energy emission through the elongated body 202 may be halted immediately. It is understood that the term “halted” may be used to issue an error code to the operator for a manual shutdown or an auto-system shut-off.

Additionally, the pressure sensor may be present anywhere within the pressure pathway, wherein the pressure pathway defines a path beginning at the generator 210 and ending at the balloon 102. In some embodiments, the pressure sensor may be within the generator 210. According to some embodiments, the pressure sensor may be within a hub 214, which is the intermediary component connecting the elongated body 202 to the generator 210 (in embodiments including a separate generator 210). The pressure sensor may be present within the elongated body 202. In some embodiments, as described in the preceding paragraph, the IVL balloon 102. The pressure sensor may be present outside of these distinct components (generator 210, hub 214, elongated body 202) but within the pressure pathway.

Furthermore, in some embodiments, the pressure sensor may be present anywhere within the IVL system 10, including outside of the previously described pressure pathway. This could include a separate device outside of the medical device 12, such as an inflation device which is either a part of, or attached to, a hub connector. This inflation device may be adjacent, but outside of, the guidewire lumen. The pressure sensor may be a part of or attached to, such an inflation device.

Additionally, a fiber interrogation mechanism (perhaps the fiber interrogation mechanism 424 as seen in FIG. 4) may be present. According to some embodiments, the purpose of the fiber interrogation mechanism is to sense or detect if the optical fiber, or at least one of the optical fibers, has broken, or in other ways become disconnected. This may be achieved by reflecting at least a portion of the energy back down the optical fiber once a pulse has been emitted, and any obstruction of this return pulse would indicate to the clinician that something has gone awry, and the IVL balloon 102 should be retrieved, and the issue fixed.

FIG. 4 illustrates a block diagram of the laser energy source system 402, according to some embodiments. As can be seen by the dotted line surrounding a majority of the components, the laser energy source system 402 includes an energy source. Power, such as power from a wall, as shown by the arrow leading through the 120 V (IN), may be provided to the power supply 302 within the energy source. The power supply 302 provides power to a flashlamp power supply 404 and a central processing unit (CPU) 418. The flashlamp power supply 404 may be controlled by the CPU 418.

The CPU 418 includes a user interface, which may involve tactile buttons and switches or other means of user communication, such as a touch screen. A power on switch 416 is shown in electronic communication with the CPU 418, as well as push buttons 420 for resetting the CPU 418 (reset) and initiating the treatment once the elongated body 202 is in place (therapy). The CPU 418 also controls the lamps 414 (On, RDY (Ready), E (Emission), and F (Fault)). The on lamp 414 indicates that the laser energy source system 402 is turned on. The RDY lamp 414 indicates that the laser energy source system 402 is connected and ready to actuate the laser energy. The E lamp 414 indicates that the laser energy is currently active. The F lamp 414 indicates that a fault has occurred, and the laser energy source system 402 needs to be reset. In IVL systems 10, including safety features such as a safety pressure sensor 422 as described above, the CPU 418 receives this feedback from the pressure sensor 422, which, as it is located in the IVL balloon 102, exists outside of the energy source.

The flashlamp power supply 404 includes lamp leads 406 that electrically couple the flashlamp power supply 404 to a laser head 408. The laser head 408 is aimed at a shutter 410, which is in electronic communication with and controlled by the CPU 418. The shutter 410 is an additional safety to prevent premature emission of the laser through the elongated body 202. The shutter 410 is commanded by the CPU 418 just prior to triggering the flashlamp, which initiates the laser energy. In the case of a laser source such as an excimer laser, the trigger for the shutter 410 may be a high-voltage switch and not a flashlamp. The shutter 410 separates the laser head 408 from the optical fibers, as indicated by the fiber out 412. The optical fibers then travel the length of the elongated body 202 to the treatment site. In IVL systems 10, including safety features such as a fiber interrogation mechanism 424 described above, the CPU 418 receives feedback from the optical fiber through the fiber interrogation mechanism 424, as shown. Because the fiber interrogation mechanism 424 may operate from anywhere along the fiber line (a break anywhere in the line can be detected anywhere else along the line, as long as the detection is occurring prior to the break), the fiber interrogation mechanism 424 is shown as being conveniently located within the energy source.

FIG. 5 illustrates a perspective view of an actuator 502 communicatively coupled to a hub 214, according to some embodiments. The hub 214 may additionally be communicatively coupled to the elongated body 202 and the generator 210. The actuator 502 is capable of communicating with the generator 210 such that, when the actuator 502 is depressed (or toggled, etc.), the generator 210 sends a pulse, or multiple pulses, of energy through the optical fibers.

Prior art systems in the IVL space include the ability to manually activate an actuator in order to deliver single pulses of energy through an optical fiber, as well as the ability to hold the actuator down in a depressed (activated) state in order to continually deliver pulses of energy. However, these prior art systems still depend on the manual activation of the device during operation, thus causing the operator to focus partial attention, as well as at least one of their hands, on the actuator itself.

In some embodiments of the present disclosure, the actuator 502 is capable of both manual activation for selectively sending pulses of energy down the optical fibers, as well as holding down (continually activating) the actuator 502 in order to deliver a continual stream of pulses to down the optical fibers. The rate at which the pulses are sent may be predetermined by the type of elongated body 202 coupled to the generator 210.

In addition, some embodiments of the present disclosure permit a hands-free operation of the actuator 502. In these embodiments, the operator may double-click the actuator 502 in quick succession in order to command the generator 210 to deliver the continual stream of pulses down the optical fibers. This may be the same frequency of pulses as those delivered through the continuous activation of the actuator 502 as described in the preceding paragraph, or a distinct frequency of pulses. The frequency at which an operator must activate the actuator 502 in order to enter this continual pulse mode may be selected so as not to interfere with the operator selectively sending pulses down the optical fibers (i.e., the frequency that the operator selectively activates the actuator 502 may be lower than the frequency needed to place the generator 210 in continual pulse mode.)

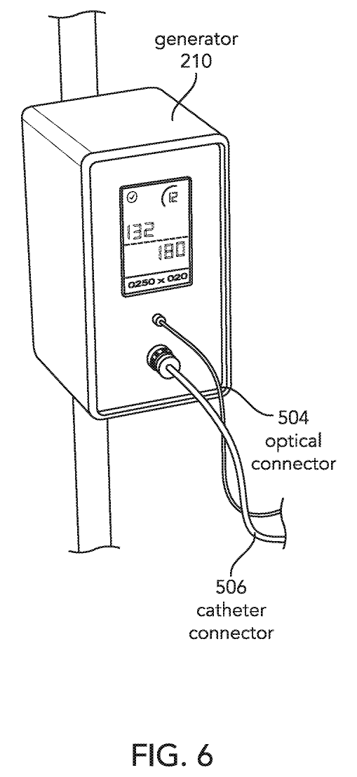

Also shown in FIG. 5 are an optical connector 504 and a catheter connector 506 coupled to the actuator 502. The optical connector 504 and the catheter connector 506 may detachably couple to the generator 210, as shown in FIG. 6. In some embodiments, the optical connector 504 effectively couples an optical fiber (or optical fibers) to the generator 210. According to some embodiments, the catheter connector 506 effectively couples the elongated body 202 to the generator 210.

Prior art systems in the IVL space include additional cables (or dongles) for coupling a catheter to a generator. This is onerous, as it requires the use of an additional sterile sleeve in order to maintain a sterile environment. Additionally, the fact that this additional cable (or dongle) is reusable is forgotten, as the catheters themselves are disposable, so this supplementary component is frequently, accidentally, disposed of by the operator with the catheter, thereby increasing the overall costs of the operations for replacement cables.

In embodiments of the present disclosure wherein the elongated body 202 may couple directly to the generator 210, these superfluous features are rendered unnecessary. No sterile sleeve or supplemental cable (or dongle) is needed to couple the elongated body 202 to the generator 210, thereby decreasing material costs in both the need for fewer materials per operation, and the elimination of components that may be incidentally disposed of with the catheter once an operation has been completed.

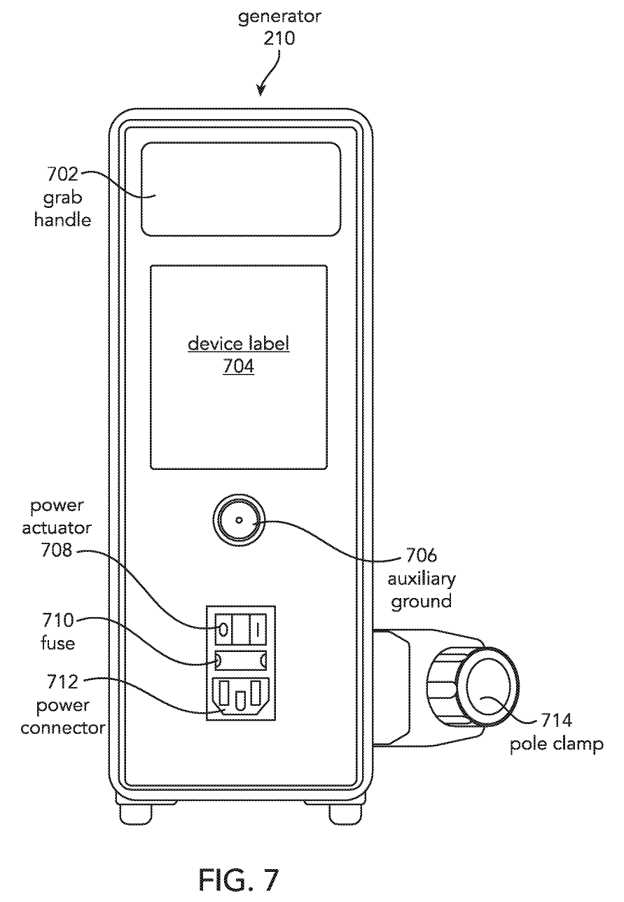

FIG. 7 illustrates an example back view of a generator 210. In some embodiments, the generator 210 includes a grab handle 702 for maneuvering the generator 210 about, such as by carrying, or rolling the generator 210 (in embodiments of the generator 210 including wheels). Also seen in FIG. 7 is a pole clamp 714 coupled to the generator 210. The pole clamp 714 may permit the operator to couple the generator 210 to a pole, such as an IV pole.

Also illustrated in FIG. 7 is an area of the generator 210 that may be dedicated to a device label 704. The device label 704 may communicate information about the generator 210 and elongated bodies 202 intended to or capable of coupling to the generator 210, as well as general safety guidelines and information. In addition, the elongated body 202 may communicate such information. The device label 704 and/or the elongated body 202 may include information or warnings including, but not exclusive to, “non-pyrogenic,” “keep away from sunlight,” “do not reuse,” “keep dry,” “recommended introducer sheath,” “recommended guidewire,” “balloon working length,” “range of atmospheric pressure to which the medical device can be safely exposed,” “defibrillation proof type BF applied part,” “do not use if package is damaged and consult instructions for use,” “caution,” “consult instructions for use,” “patents,” “use-by date,” “batch code,” “the manufacturer’s serial number code so that the specific device can be identified,” “range of humidity to which the medical device can be safely exposed,” “manufacturer,” “do not resterilize,” “sterilized using ethylene oxide,” “caution: USA Federal law restricts these devices to sale by, or on the order of a physician or licensed practitioner,” “balloon diameter,” “catalog number,” “medical device was manufactured YYY-MM-DD,” “temperature limits to which the medical device can be safely exposed,” and “projectile hazard: keep out of MR scanner rooms.” Each of these pieces of information or warnings may additionally be accompanied by a symbol, or image depicting what the information or warning is detailing.

The generator 210 may further include an auxiliary ground 706, for grounding the generator 210 as a whole. In some embodiments, the generator 210 includes a power actuator 708 for turning the generator 210 on and off. According to some embodiments, the generator 210 includes a fuse 710 and a power connector 712 for attaching the generator 210 to alternating current (AC) power.

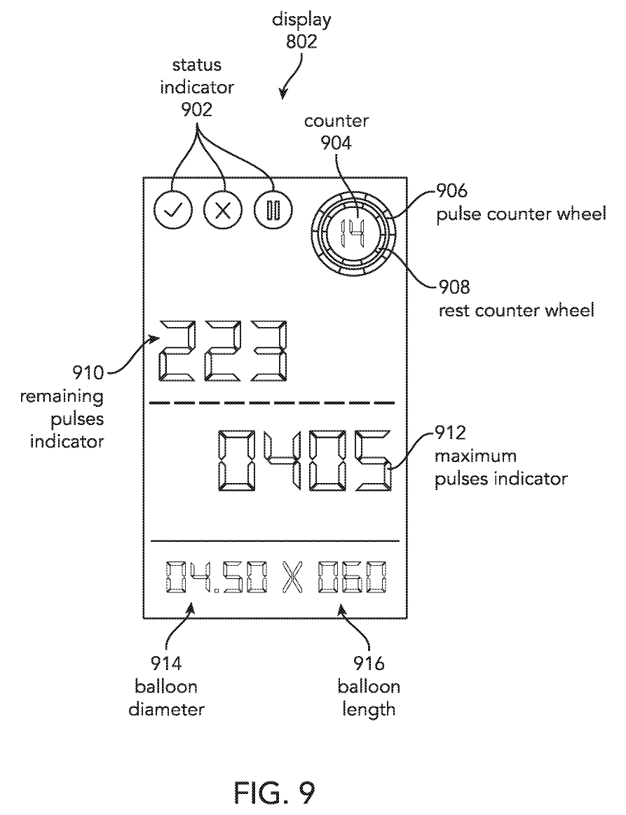

Devices in the prior art include batteries, providing direct current (DC) power to the generator. Because of this, it is warranted for these prior art devices to somehow communicate how much life the battery still has, and thus this communication takes up valuable real estate on any provided display screen. The use of AC power in the present disclosure may eliminate the need to communicate this information about battery life, and as such, additional information may be communicated to an operator, as detailed in FIG. 9 below.

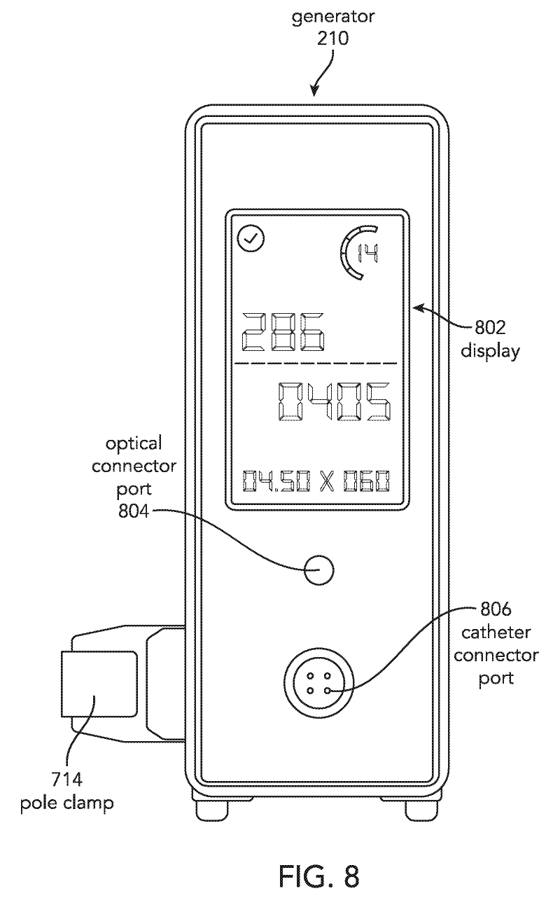

FIG. 8 illustrates an embodiment front view of a generator 210, perhaps the generator 210 of FIG. 7. FIG. 8 includes the pole clamp 714 as previously discussed in FIG. 7. Also seen in FIG. 8 is a display 802, for which greater details are supplied in the discussion of FIG. 9. FIG. 8 also illustrates an optical connector port 804 and a catheter connector port 806. The optical connector 504 as shown and described in FIGS. 5 and 6 may detachably couple to the generator 210 via the optical connector port 804. Likewise, the catheter connector 506 as shown and described in FIGS. 5 and 6 may detachably couple to the generator 210 via the catheter connector port 806.

In some embodiments, the display 802 is communicatively coupled to the generator 210. The display 802 may be interacted with and/or viewable from a remote computing device, such as through a smartphone application. Additionally or alternatively, the display 802 may be electrically coupled to the generator 210. According to some embodiments, the display 802 is mechanically coupled to the generator 210. In additional embodiments, the display 802 is integrated into the generator 210.

In some embodiments, the generator 210 includes passthrough circuitry enabling the powering of external devices, such as through a charging port. In additional or alternative embodiments, the generator 210 includes wireless charging capabilities configured to provide energy to devices as an additional method of passthrough charging.

FIG. 9 illustrates the display 802 of FIG. 9, according to some embodiments. As previously discussed in FIG. 8, the use of AC power renders the presentation of battery life on such a display 802 unnecessary, leaving room for other, additional information that an operator may find pertinent. For example, status indicators 902 are provided near the top left of the display 802, though it is understood that these status indicators 902, and any other features provided on such a display 802 could be moved to any location as desired.

These status indicators 902 may include a checkmark symbol, or an equivalent symbol, indicating that the IVL system 10 is enabled and ready to provide laser energy through the optical fibers. These status indicators 902 may also include an “X” symbol, or an equivalent symbol, indicating that there is a condition requiring attention to restore use of the IVL system 10, or identifying a problem with the IVL system 10. In the case where there may be a problem with the IVL system 10, an error code may replace the numeric digits in the location of the remaining pulses indicator 910. Additionally, these status indicators 902 may include a set of parallel lines, internationally recognized as a “pause” symbol, or an equivalent symbol, indicating that the IVL system 10 is in a rest period (or cooldown period) or awaiting other actions in order to return to functionality. The rest period may be a predetermined amount of time or selectable by an operator. Any or all of the status indicators 902 may further be color coordinated to facilitate quick understanding of the information being provided.

The display 802 may also include a counter 904, shown in FIG. 9 in the top left of the display 802. The counter 904 may count upward, indicating the number of pulses that have been sent in a sequence. As treatments may be broken up into multiple areas of treatment (or sites within a specific area), this counter 904 may indicate when a specific location's treatment has been completed and it is time to move to the next treatment site. Additionally, when the IVL system 10 enters a rest period as previously described, the counter 904 may count down, in seconds or other measures of time, the amount of time left in such a rest period.

Additionally, any of the indicators may further serve to act as an indication of when a pulse is occurring. Any one of the status indicator 902, counter 904, and pulse counter wheel 906 may flash in response to a delivered pulse, visually indicating to an operator when this pulse occurs. An audio signal, which may include an audio notification, an audio indication, an audio warning, an audio alert, and the like, may signal the occurrence of a pulse as well, either in addition to or as an alternative to the visual signal, which may include a visual notification, a visual indication, a visual warning, a visual alert, and the like. In some embodiments, this audio signal may not occur at the same frequency as the pulses, but rather be sounded to indicate a start of pulsing, an end of pulsing, and/or an elongated noise for the entire duration of pulsing.

In some embodiments, the display 802 includes a pulse counter wheel 906 and a rest counter wheel 908. In addition to the counter 904 as described in the previous paragraph, a pulse counter wheel 906 may visually indicate the amount of pulses that have been delivered in a sequence. This pulse counter wheel 906 may act as a type of “progress bar,” and fill the circle, either clockwise or counter-clockwise, from empty to completely full. Similarly, the rest counter wheel 908 may act in an inverse manner, slowly depleting from a full circle to an empty circle, matching the counter 904 counting down the amount of time left in a period of rest.

According to some embodiments, the display 802 includes a remaining pulses indicator 910, as well as a maximum pulses indicator 912. The remaining pulses indicator 910 may indicate how many pulses are left before the attached elongated body 202 reaches its end-of-life and can no longer be used, or, alternatively, the number of pulses remaining in a treatment. The maximum pulses indicator 912 may indicate how many pulses an elongated body 202 can deliver, from its first use until its end-of-life. As detailed previously, in the event an error code is triggered, the error code may replace the remaining pulses indicator 910. Both the remaining pulses indicator 910 and the maximum pulses indicator 912 may be populated at the time an elongated body 202 is coupled to the generator 210. The elongated body 202 may include a chip, or programmable read-only memory (PROM) for storing this information, thereby informing the generator 210 if the remaining pulses indicator 910 should be lower than its initial value. The maximum pulses indicator 912, as it is set to a specific number, may not change.

Either or both of the remaining pulses indicator 910 and the maximum pulses indicator 912 may count incrementally, changing the number in response to each delivered pulse. In some embodiments, the pulses occur quickly enough that displaying the entire number may be incoherent to an operator at a glance as the number is changing too quickly. In such embodiments, the last digit may be replaced with an “X” or omitted entirely. As shown in FIG. 9, for example, the remaining pulses indicator 910 indicates 223 pulses and the maximum pulses indicator 912 indicates 405 pulses. In some embodiments, this would be represented by the remaining pulses indicator 910 indicating 22X pulses, and the maximum pulses indicator 912 indicating 40X pulses, with these numbers only incrementing every 10 pulses, thus providing a clearer indication of the numeric value to an operator at a glance.

The display 802 may further provide a warning to the operator, either visual, audio, or both, when either of the remaining pulses indicator 910 and/or the maximum pulses indicator 912 approaches zero.

While previously disclosed as a PROM, it is understood that the system is capable of utilizing any type of read-only memory (ROM), including but not limited to masked read-only memory (MROM) (for instances where new information does not need to be written to the ROM), erasable programmable read-only memory (EPROM) (for instances where the user wants to be able to overwrite already existing or previously written instructions), Flash EPROM, and electrically erasable programmable read-only memory (EEPROM)(for increased speed of erasing and writing to the memory).

The PROM may also be a part of the printed circuit board assembly (PCBA) within the enclosure of the actuator 502. In some embodiments, the generator 210 is configured to write information to the PROM. Additionally or alternatively, the generator 210 may be configured to read information from the PROM.

As illustrated at the bottom of the display 802 in FIG. 9, indicators of the size of the components of the medical device 12 may be present. In this embodiment, a balloon diameter 914 and a balloon length 916 are included. Any dimensions desired may be presented in this manner. Additionally, the PROM may also carry this information, and it may be automatically presented on the display 802 when the elongated body 202 is connected to the generator 210.

While not specifically illustrated in FIG. 9, many more indicators may be present, either in combination with or in lieu of those mentioned above. These indicators may include, but are not limited to, pressure (such as real-time pressure in atmospheres (Atm), pascals (Pa), Torr, inches of mercury (Hg), millibars (mb), or pound-force per square inch (lbf/in2, or, psi)). These indicators may also include different thresholds of pressure (nominal or near-rated burst pressure, or treatment pressure).

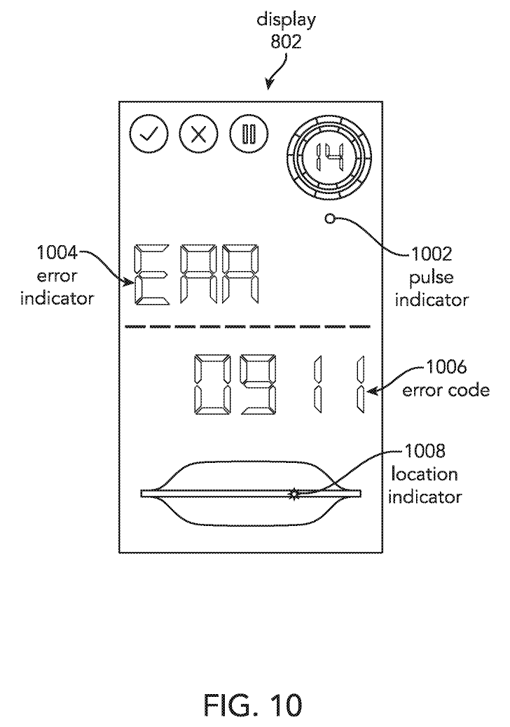

In some embodiments, including either longitudinally translatable or non-translatable electrodes, these indicators may include a location of treatment within the balloon (as shown and disclosed in FIG. 10 below). This may include the location of the translatable electrodes as they move through the balloon, or the location of the balloon through the location of the non-translatable electrodes within the balloon. This information may be presented through a diagram of the balloon showing the electrodes' locations, and may also indicate, via the diagram, each pulse of the electrodes. According to some embodiments, the indicators may include the introducer size.

The indicators may also include a status of the treatment (either current progress of the treatment, or an indication of whether the treatment is completed or not). This may occur through an image or depiction of the vessel opening as treatment progresses. In some embodiments, this serves as a general progress bar for the treatment. In other embodiments, the system is capable of detecting the level of calcification in the treatment area, such as through the use of piezo crystals in and/or on the balloon, and reports that information in real-time back to the PROM to provide an accurate, live update of the progress of the treatment.

Specifically, FIG. 10 illustrates another embodiment of the display 802, wherein the remaining pulses indicator 910 is replaced by an error indicator 1004. As seen in FIG. 10, the segmental display (in this embodiment shown as a seven-segment display) changes from numbers to “ERR,” indicative that an error has occurred. Also shown in FIG. 10 is the maximum pulses indicator 912 replaced by an error code 1006. When an error indicator 1004 appears on the display 802, an operator may look to the error code 1006 to see what exactly has gone wrong. An audio alert may also be used in addition to or as an alternative to the error indicator 1004. This audio alert may be of a different frequency than other audio disclosed herein, such as when the system is pulsing. It should be appreciated that the audio alert may include an audio signal, an audio notification, an audio indication, an audio warning, and the like.

The following are some examples of what may trigger an error code. In some embodiments, the error is a shattering of the optical fiber. According to some embodiments, the error is a change in photon radiation along an optical pathway. The error may be a change in an amount of optical power. In some embodiments, the error is a pulse width outside of a nominal range. According to some embodiments, the error is a breaking of the optical fiber.

The error may be an undesired increase in a temperature of a laser head. In some embodiments, the error is an ultrasound intensity outside of a nominal range. According to some embodiments, the error is a pressure of a pulse outside of a nominal range. The error may be a humidity of an ambient environment outside of a nominal range. In some embodiments, the error is an acceleration experienced by the generator that is higher than a predetermined maximum acceleration.

According to some embodiments, the error is an increased amount of gas in the inflation fluid. The error may be a pulse energy outside of a nominal range. In some embodiments, the error is a peak power outside of a nominal range.

According to some embodiments, the error is an inflation pressure of the balloon outside of a nominal range. The error may be a temperature relating to the system outside of a nominal range. In some embodiments, the error is an ambient temperature outside of a nominal range. According to some embodiments, the error is a temperature of the inflation fluid outside of a nominal range. The error may be a temperature of an optical fiber connector outside of a nominal range.

In some embodiments, the error is a flow rate of coolant through the generator outside of a nominal range. According to some embodiments, the error is a conductivity of the coolant outside of a nominal range. The error may be a temperature of the coolant outside of a nominal range. In some embodiments, the error is a pressure of the coolant outside of a nominal range.

According to some embodiments, the error is leakage from a high reflector. The error may be a voltage outside of a nominal range.

Also shown in FIG. 10 is a pulse indicator 1002. This pulse indicator 1002 may be used in addition to or as an alternative to the visual indications of a pulse as described above with respect to the status indicator 902, counter 904, and pulse counter wheel 906. The pulse indicator 1002 may flash in response to a delivered pulse, visually indicating to an operator when the pulse occurs. In some embodiments, the pulse indicator 1002 may flash in response to a delivered pulse, visually indicating to an operator when each pulse occurs. According to some embodiments, the pulse indicator 1002 may remain lit for the duration of a treatment. An audio signal may be provided to signal the occurrence of a pulse as well, either in addition to or as an alternative to the visual signal provided by the pulse indicator 1002.

FIG. 10 also illustrates a location indicator 1008. The location indicator 1008 may take the appearance of a balloon along with the portion of the catheter extending through the balloon. As shown in FIG. 10, the location indicator 1008 shows the location of an electrode through a visual representation, which may include a flashing or strobing effect. The flashing or strobing visualization of the electrode may occur in response to a delivered pulse, visually indicating to an operator when each pulse occurs. An audio signal may be provided to signal the occurrence of a pulse as well, either in addition to or as an alternative to the visual signal provided by the location indicator 1008. In additional or alternative embodiments, the visual representation includes a constantly lit point indicative of the position of an electrode.

In some embodiments, the electrodes are translatable along the catheter, and the location indicator 1008 may indicate the location of these translatable electrodes as they move through the balloon. In additional or alternate embodiments, the electrodes are non-translatable, and the location indicator 1008 may provide information to the operator about the location of the balloon via the location of the non-translatable electrodes. In some embodiments, the electrodes may be rotated about a central longitudinal axis of the catheter, and the location indicator 1008 may indicate where the electrode is with respect to this central longitudinal axis. In further embodiments, the electrodes may be deflected away from the catheter, and the location indicator 1008 may indicate where the electrode is with respect to the catheter. Any combination of the above location indicator 1008 embodiments may be expressed in tandem on the display 802.

In any of the embodiments of the display 802 above, the display may be detachable, such that an operator may place the display in a more convenient location for processing the provided information. This location may be anywhere within the room that is within sight of the operator while performing an operation, such as on the drapage of the patient. Additionally, the display 802 may be rotatably coupled to an item such as a rolling pole, so that the operator may move the display 802 as necessary to keep it within their line of sight.

Additionally, any of the indicators as detailed in FIGS. 9 and 10 above, including the status indicators 902, counter 904, pulse counter wheel 906, rest counter wheel 908, remaining pulses indicator 910, maximum pulses indicator 912, balloon diameter 914, balloon length 916, pulse indicator 1002, error indicator 1004, error code 1006, and location indicator 1008 may include colors to orient an operator as to what information they are looking at more quickly.Devices in the prior art are limited in the information provided to the operator. The information an operator might garner from a prior art device includes the remaining battery life (due to the use of DC power), the dimensions of the balloon 102 of the balloon catheter, and the number of pulses delivered. This causes the operator to need to perform calculations in their head during an operation to know how far into an operation they are, as there is no indication of the number of pulses left – either for a treatment cycle or the catheter life expectancy as a whole.

Through providing the number of pulses a catheter has remaining in its life, the number of pulses remaining for a specific treatment cycle, and the time between subsequent treatment cycles (the aforementioned cooldown period), this burden is taken off the operator, as they can know glean this information at a glance of the display 802 on the generator 210.

As disclosed in FIG. 8, any of the aforementioned components of the display 802 may be implemented in a remote computing device, such as a software application run on a smartphone, such that the operator may view any component of the display 802 from such a remote computing device.

In additional embodiments, the information provided by the display 802 is capable of running through a non-transitory computer-readable media executable on a processor of a device, such as a remote computing device. In such embodiments, the non-transitory computer-readable media may display, on the device, the information such that an operator may view any component of the display 802.Included in the present disclosure is an apparatus, including a generator configured to provide electricity to a catheter. In some embodiments, the apparatus includes a display coupled to the generator, the display including a status indicator.

According to some embodiments, the catheter is an intravascular lithotripsy (IVL) catheter. The display may be coupled to a remote computing device. In some embodiments, the status indicator is displayed via a software application running on the remote computing device.

According to some embodiments, the status indicator is configured to indicate the apparatus is in a ready state. In some embodiments, the status indicator is configured to indicate the apparatus is in a rest state. The status indicator may be configured to indicate the apparatus is experiencing an error.

According to some embodiments, the display includes a counter. The counter may be configured to count down a number of remaining pulses in a treatment cycle. In some embodiments, the counter is configured to count down a remaining amount of time in a cooldown period.

According to some embodiments, the display includes a pulse counter wheel. The pulse counter wheel may be configured to visually indicate a number of remaining pulses in a treatment cycle. In some embodiments, the pulse counter wheel includes a progress bar. According to some embodiments, the pulse counter wheel is circular.

The display may include a rest counter wheel. In some embodiments, the rest counter wheel is configured to visually indicate a remaining amount of time in a cooldown period. According to some embodiments, the rest counter wheel includes a progress bar. The rest counter wheel may be circular.

In some embodiments, the display includes a remaining pulses indicator. According to some embodiments, the remaining pulses indicator is configured to count down a number of remaining pulses in a life expectancy of the catheter.

The display may include a maximum pulses indicator. In some embodiments, the maximum pulses indicator is configured to indicate a total number of pulses in a life expectancy of the catheter.

According to some embodiments, the display includes a size indicator. The size indicator may include a balloon diameter. In some embodiments, the size indicator includes a balloon length.

Also included in the present disclosure is a system, including an elongated body having an inner elongated structure including a guidewire lumen. In some embodiments, the system includes an erasable programmable read-only memory (EPROM) configured to store information. According to some embodiments, the system includes a balloon positioned at a distal portion of the elongated body, the balloon configured to be inflated. The system may include an optical fiber positioned along the elongated body and within the balloon, the optical fiber configured to emit pressure waves to fragment a buildup in an organ. In some embodiments, the system includes a generator configured to provide energy to the optical fiber, the generator including a display configured to show the information. According to some embodiments, the elongated body is configured to couple to the generator, thereby forming an optical pathway between the generator and the optical fiber.

The generator may be configured to receive power through a direct current (DC) power supply. In some embodiments, the generator includes a female connector, and the elongated body includes a male connector. According to some embodiments, the male connector is configured to couple to the female connector, thereby coupling the elongated body to the generator.

The display may include a status indicator. In some embodiments, the status indicator is configured to indicate the system is in a ready state. According to some embodiments, the status indicator is configured to indicate the system is in a rest state. The status indicator may be configured to indicate the system is experiencing an error.

The display may include a counter. In some embodiments, the counter is configured to count down a number of remaining pulses in a treatment cycle. According to some embodiments, the counter is configured to count down a remaining amount of time in a cooldown period.

The display may include a pulse counter wheel. In some embodiments, the pulse counter wheel is configured to visually indicate a number of remaining pulses in a treatment cycle. According to some embodiments, the pulse counter wheel includes a progress bar. The pulse counter wheel may be circular.

In some embodiments, the display includes a rest counter wheel. According to some embodiments, the rest counter wheel is configured to visually indicate a remaining amount of time in a cooldown period. The rest counter wheel may include a progress bar. In some embodiments, the rest counter wheel is circular.

According to some embodiments, the display includes a remaining pulses indicator. The remaining pulses indicator may be configured to count down a number of remaining pulses in a life expectancy of the elongated body.

In some embodiments, the display includes a maximum pulses indicator. According to some embodiments, the maximum pulses indicator is configured to indicate a total number of pulses in a life expectancy of the elongated body.

The display may include a size indicator. In some embodiments, the size indicator includes a balloon diameter. According to some embodiments, the size indicator includes a balloon length.

The system may include an actuator, the actuator configured to cause the generator to provide electricity to the emitter. In some embodiments, the actuator is configured to send a single pulse to the emitter in response to being activated once. According to some embodiments, the actuator is configured to send a continual stream of pulses to the emitter in response to being activated continuously. The actuator may be configured to send a continual stream of pulses to the emitter in response to being activated twice in quick succession.

In some embodiments, the generator is configured to read the information. According to some embodiments, the generator is configured to write the information.

Also included in the present disclosure is a system, including an elongated body having an inner elongated structure including a guidewire lumen. In some embodiments, the system includes a balloon positioned at a distal portion of the elongated body, the balloon configured to be inflated. According to some embodiments, the system includes an optical fiber positioned along the elongated body and terminating within the balloon, the optical fiber configured to emit pressure waves to fragment a buildup in an organ. The system may include a generator configured to provide energy through the optical fiber, the generator configured to receive power through a direct current (DC) power supply, the generator including a display. In some embodiments, the elongated body is configured to couple to the generator, thereby forming an optical pathway between the generator and the optical fiber.

According to some embodiments, the generator includes a female connector, and the elongated body includes a male connector. The male connector may be configured to couple to the female connector, thereby coupling the elongated body to the generator.

In some embodiments, the system further includes an erasable programmable read-only memory (EPROM) configured to store information. According to some embodiments, the generator is configured to read the information. The generator may be configured to write the information.

In some embodiments, the display includes a status indicator. According to some embodiments, the status indicator is configured to indicate the system is in a ready state. The status indicator may be configured to indicate the system is in a rest state. In some embodiments, the status indicator is configured to indicate the system is experiencing an error.

According to some embodiments, the display includes a counter. The counter may be configured to count down a number of remaining pulses in a treatment cycle. In some embodiments, the counter is configured to count down a remaining amount of time in a cooldown period.

According to some embodiments, the display includes a pulse counter wheel. The pulse counter wheel may be configured to visually indicate a number of remaining pulses in a treatment cycle. In some embodiments, the pulse counter wheel includes a progress bar. According to some embodiments, the pulse counter wheel is circular.

The display may include a rest counter wheel. In some embodiments, the rest counter wheel is configured to visually indicate a remaining amount of time in a cooldown period. According to some embodiments, the rest counter wheel includes a progress bar. The rest counter wheel may be circular.

In some embodiments, the display includes a remaining pulses indicator. According to some embodiments, the remaining pulses indicator is configured to count down a number of remaining pulses in a life expectancy of the elongated body.

The display may include a maximum pulses indicator. In some embodiments, the maximum pulses indicator is configured to indicate a total number of pulses in a life expectancy of the elongated body.

According to some embodiments, the display includes a size indicator. The size indicator may include a balloon diameter. In some embodiments, the size indicator includes a balloon length.

According to some embodiments, the system further includes an actuator, the actuator configured to cause the generator to provide electricity to the emitter. The actuator may be configured to send a single pulse to the emitter in response to being activated once. In some embodiments, the actuator is configured to send a continual stream of pulses to the emitter in response to being activated continuously. According to some embodiments, the actuator is configured to send a continual stream of pulses to the emitter in response to being activated twice in quick succession.

Also included in the present disclosure is a system, including an elongated body having an inner elongated structure including a guidewire lumen. In some embodiments, the system includes a balloon positioned at a distal portion of the elongated body, the balloon configured to be inflated. According to some embodiments, the system includes an optical fiber positioned along the elongated body and terminating within the balloon, the optical fiber configured to emit pressure waves to fragment a buildup in an organ. The system may include a generator configured to provide energy through the optical fiber, the generator configured to receive power through a direct current (DC) power supply, the generator including a display. In some embodiments, the elongated body is configured to couple to the generator, thereby forming an optical pathway between the generator and the optical fiber.

According to some embodiments, the generator includes a female connector, and the elongated body includes a male connector. The male connector may be configured to couple to the female connector, thereby coupling the elongated body to the generator.

In some embodiments, the system further includes a programmable read-only memory (PROM) configured to store information. According to some embodiments, the generator is configured to perform an action selected from the group consisting of reading the information, writing the information, and combinations thereof.

The display may include a status indicator. In some embodiments, the status indicator is configured to indicate information about the system selected from the group consisting of the system is in a ready state, the system is in a rest state, the system is experiencing an error, and combinations thereof.

According to some embodiments, the display includes a counter. The counter may be configured to count in a direction selected from the group consisting of upward, downward, and combinations thereof. In some embodiments, the counter is configured to count a metric selected from the group consisting of a number of remaining pulses in a treatment cycle, a remaining amount of time in a cooldown period, and combinations thereof.

According to some embodiments, the display includes a counter wheel selected from the group consisting of a pulse counter wheel, a rest counter wheel, and combinations thereof. The pulse counter wheel may be configured to visually indicate a number of remaining pulses in a treatment cycle. In some embodiments, the rest counter wheel is configured to visually indicate a remaining amount of time in a cooldown period.

According to some embodiments, the counter wheel includes a progress bar. The display may include a remaining pulses indicator configured to count down a number of remaining pulses in a life expectancy of the elongated body. In some embodiments, the display includes a maximum pulses indicator configured to indicate a total number of pulses in a life expectancy of the elongated body. According to some embodiments, the display includes a size indicator, the size indicator including a size metric selected from the group consisting of a balloon diameter, a balloon length, and combinations thereof.

The system may further include an actuator, the actuator configured to cause the generator to provide electricity to the emitter. In some embodiments, the actuator is configured to send a single pulse to the emitter in response to being activated once. According to some embodiments, the actuator is configured to send a continual stream of pulses to the emitter in response to being activated once. The actuator may be configured to stop the continual stream of pulses to the emitter in response to being activated a subsequent time.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus,

such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a predetermined desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that these and similar terms are to be associated with the appropriate physical quantities and are merely convenient labels applied to these quantities. The present disclosure can refer to the action and processes of a computing system, or similar electronic computing device, that manipulates and transforms data represented as physical (electronic) quantities within the computing system's registers and memories into other data similarly represented as physical quantities within the computing system memories or registers or other such information storage systems.

The present disclosure also relates to an apparatus for performing the operations herein. This apparatus can be specially constructed for the intended purposes, or it can include a general-purpose computer selectively activated or reconfigured by a computer program stored in the computer. Such a computer program can be stored in a computer-readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computing system bus.

The algorithms and displays presented herein are not inherently related to any particular computer or other apparatus. Various general-purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product, or software, that can include a machine-readable medium having stored thereon instructions, which can be used to program a computing system (or other electronic devices) to perform a process according to the present disclosure. A machine-readable medium includes any mechanism for storing information in a form readable by a machine (e.g., a computer). In some embodiments, a machine-readable (e.g., computer-readable) medium includes a machine (e.g., a computer) readable storage medium such as a read only memory (“ROM”), random access memory (“RAM”), magnetic disk storage media, optical storage media, flash memory components, etc.

In the foregoing specification, embodiments of the disclosure have been described with reference to specific example embodiments thereof. It will be evident that various modifications can be made thereto without departing from the broader spirit and scope of embodiments of the disclosure as set forth in the following claims. The specification and drawings are, accordingly, to be regarded in an illustrative sense rather than a restrictive sense.

We claim:

1. A system, comprising:
an elongated body comprising an inner elongated structure including a guidewire lumen;
a balloon positioned at a distal portion of the elongated body, the balloon configured to be inflated;
an optical fiber positioned along the elongated body and terminating within the balloon, the optical fiber configured to generate pressure waves to fragment a buildup in an organ; and
a generator configured to provide energy through the optical fiber, the generator configured to receive power through a direct current (DC) power supply, the generator comprising a display,
wherein the elongated body is configured to couple to the generator, thereby forming an optical pathway between the generator and the optical fiber.

2. The system of claim 1, the generator comprising a female connector, and the elongated body comprising a male connector,
wherein the male connector is configured to couple to the female connector, thereby coupling the elongated body to the generator.

3. The system of claim 1, further comprising a programmable read-only memory (PROM) configured to store information.

4. The system of claim 3, wherein the generator is configured to perform an action selected from the group consisting of reading the information, writing the information, and combinations thereof.

5. The system of claim 1, the display comprising a status indicator.

6. The system of claim 5, wherein the status indicator is configured to indicate information about the system selected from the group consisting of the system is in a ready state, the system is in a rest state, the system is experiencing an error, and combinations thereof.

7. The system of claim 1, the display comprising a counter.

8. The system of claim 7, wherein the counter is configured to count in a direction selected from the group consisting of upward, downward, and combinations thereof.

9. The system of claim 8, wherein the counter is configured to count a metric selected from the group consisting of a number of remaining pulses in a treatment cycle, a remaining amount of time in a cooldown period, and combinations thereof.

10. The system of claim 1, the display comprising a counter wheel selected from the group consisting of a pulse counter wheel, a rest counter wheel, and combinations thereof.

11. The system of claim 10, wherein the pulse counter wheel is configured to visually indicate a number of remaining pulses in a treatment cycle.

12. The system of claim 10, wherein the rest counter wheel is configured to visually indicate a remaining amount of time in a cooldown period.

13. The system of claim 10, the counter wheel comprising a progress bar.

14. The system of claim 1, the display comprising a remaining pulses indicator configured to count down a number of remaining pulses in a life expectancy of the elongated body.

15. The system of claim 1, the display comprising a maximum pulses indicator configured to indicate a total number of pulses in a life expectancy of the elongated body.

16. The system of claim 1, the display comprising a size indicator, the size indicator comprising a size metric selected from the group consisting of a balloon diameter, a balloon length, and combinations thereof.

17. The system of claim 1, further comprising an actuator, the actuator configured to cause the generator to provide laser energy to the optical fiber.

18. The system of claim 17, wherein the actuator is configured to send a single pulse of laser energy to the optical fiber in response to being activated once.

19. The system of claim 17, wherein the actuator is configured to send a continual stream of laser energy to the optical fiber in response to being activated once.

20. The system of claim 19, wherein the actuator is configured to stop the continual stream of pulses to the optical fiber in response to being activated a subsequent time.
